(19) United States
(12) Patent Application Publication
(10) Pub. No.: US 2026/0240487 A1
(43) Pub. Date: Aug. 20, 2026

(54) SYSTEM AND PROTOCOL FOR MONITORING PREGNANCY HEALTH

(71) Applicants: AmpX Technologies, Inc., College Park, MD (US); The United States of America, as represented by the Secretary, Department of Health and Human Servic, Bethesda, MD (US)

(72) Inventors: Amir Hossein Gandjbakhche, Bethesda, MD (US); Thien T. Nguyen, McLean, VA (US); Zeyu Zhang, College Park, MD (US); Soongho Park, McLean, VA (US); George H. Downey, Greenbelt, MD (US); Alireza Khaligh, Arlington, VA (US); Kosar Khaksari, Washington, DC (US)

(73) Assignees: AmpX Technologies, Inc., College Park, MD (US); The United States of America, as represented by the Secretary, Department of Health and Human Servic, Bethesda, MD (US)

(21) Appl. No.: 19/162,323

(22) PCT Filed: Mar. 8, 2024
(86) PCT No.: PCT/US2024/019168

§ 371 Date: Sep. 4, 2025

Related U.S. Application Data
(60) Provisional application No. 63/451,066, filed on Mar. 9, 2023.

(51) Int. Cl. A61B 5/00; A61B 5/024; A61B 5/11; A61B 5/1455; A61B 5/1464

(52) U.S. Cl. CPC A61B 5/4356; A61B 5/0022; A61B 5/02411; A61B 5/11; A61B 5/14551; A61B 5/1464; A61B 5/7264; A61B 2503/02

(57) ABSTRACT

The disclosure provides systems and a protocol for monitoring the health of a fetus and a mother during pregnancy. A wearable device utilizing near-infrared spectroscopy (NIRS) is disclosed for monitoring a number of parameters associated with placental tissue, the fetus, and/or the mother. The device includes one or more light sources and one or more photodetectors, each photodetector separated from a particular light source by a corresponding separation distance. The device further includes a processor configured to collect data based on intensity information collected by the plurality of photodetectors responsive to activation of one or more of the plurality of light sources. The data is used to estimate an oxygen saturation level for a layer of placental tissue in a uterus of a mother, or maternal physiological signals including respiratory functions, cardiac functions, oxygenation levels, or fetal cardiac functions.

### CROSS-REFERENCE TO RELATED APPLICATIONS

This application claims the benefit of U.S. Provisional Application No. 63/451,066, filed Mar. 9, 2023, which is herein incorporated by reference in its entirety.

### STATEMENT REGARDING FEDERALLY SPONSORED RESEARCH OR DEVELOPMENT

This invention was made with Government support under project number Z01-HD000261 by the National Institutes of Health. The Government has certain rights in the invention.

### FIELD

The present disclosure relates to methods of monitoring the health of a patient during pregnancy. More specifically, the present disclosure describes an apparatus and a protocol that uses near-infrared spectroscopy (NIRS) to monitor oxygen saturation levels in placental tissue as well as other parameters related to the fetus and/or patient.

### BACKGROUND

Observing the placenta during pregnancy can offer insights into the in utero fetal environment. Variations in the size of the placenta throughout early pregnancy have been associated with placental injury from factors such as maternal malnutrition or anemia. Reduced uteroplacental perfusion is often associated with fetal growth restriction (FGR), a condition where the fetus fails to reach their genetic growth potential, and the associated condition, pre-eclampsia. In cases of pre-eclampsia, pregnant women will often have hypertension, protein in their urine, and symptoms such as blurred vision and headaches, posing significant health risks to the mother. Additionally, pre-eclampsia and FGR can increase risk for perinatal death of the fetus and premature delivery. Reduced uteroplacental perfusion can also lead to chronic hypoxia, a condition where the tissue is not oxygenated adequately, and poor fetal nutrition. These factors increase risk for cognitive impairments in the child, including cerebral palsy and lifelong metabolic outcomes. Additionally, reduced perfusion can lead to perinatal asphyxia, a lack of oxygen and blood flow to the fetus before, during, or immediately after birth. More severe cases of asphyxia, where the fetus has low oxygen levels for an extended period, may result in permanent damage to the baby's major organs, including the brain, liver, and kidneys, organ failure, or death. Therefore, monitoring placental oxygenation level and maternal physiological signals may be useful in distinguishing between a normal fetus and one with FGR and/or the associated conditions discussed above. Such signals may provide additional insight into the expected pregnancy outcome, when monitored during pregnancy. Furthermore, identification of such complications during pregnancy can allow for earlier interventions, including medications to reduce risk of perinatal mortality (e.g., sildenafil, esomeprazole, and metformin) and/or maternal gene therapy.

Fetal movement has long served as a measure for fetal well-being and nervous system development, helping to identify adverse pregnancy outcomes. A decrease in fetal movement is frequently an early warning sign of fetal health complications or stillbirths. Currently, the most common method for fetal movement measurement assessment is maternal self-count, where the mother counts the number of movements observed over a fixed time or measures the time period taken to reach a fixed number of movements. Maternal counting of fetal movements is simple and can be recorded on a regular basis. However, maternal perception of movement is subjective, hence there is no consensus on whether maternal counting improves perinatal outcomes. The other methods to assess fetal movement are technology assisted or automated. The technology assisted approach makes use of aids such as ultrasound or MRI in conjunction with health professional assessment, while the automated technology approach, such as actigraphy and/or cardiotocography, can directly assess fetal movement without input from health professionals. These technologies can detect fetal movement with high accuracy, but usually are only used during a short perinatal visit, while changes in fetal movement are often sudden. A wearable device, which can monitor fetal movements, can allow for real-time assessment of fetal well-being and assist to detect potential adverse events for a longer period.

There are many non-invasive techniques available to image the fetus and measure the fetal and placental oxygen saturation levels. One of these techniques is magnetic resonance imaging (MRI), by which it is possible to image the entire fetus and placenta at any gestational period. Although MRI is non-invasive, it is expensive and time consuming, and is not portable. MRI may not be appropriate to monitor fetal physiological signals and placental oxygenation frequently over long periods of time. As such, MRI may also not be suitable to identify and prevent conditions associated with reduced uteroplacental perfusion.

Another non-invasive measure that is typically used to assess pregnancy development is ultrasound. Utilizing point-of-care technology like ultrasound provides direct assessment of fetal development and can detect abnormalities in the fetal brain and other organs. Combining transvaginal and transabdominal ultrasound approaches has been shown to be more effective in detecting fetal abnormalities than using one method independently. However, similar to MRI, ultrasound is unable to provide continuous or frequent monitoring over long periods of time.

Therefore, there is a need in the medical field for a device that is capable of monitoring placental oxygen saturation levels, multiple additional fetal and maternal physiological signals (e.g., temperature, tissue oxygenation levels, heart rate, etc.), and movement activity of the fetus, over long periods of time, using a safe, non-invasive, and wearable device.

### SUMMARY

Embodiments of the present disclosure relate to systems and a protocol for monitoring the health of a fetus and a mother during pregnancy. A wearable device utilizing near-infrared spectroscopy (NIRS) is disclosed for monitoring a number of parameters associated with placental tissue, the fetus, and/or the mother.

In a first aspect of the present disclosure, an apparatus for monitoring placental oxygen saturation level is disclosed. The apparatus includes a light source and a photodetector. The apparatus further includes at least one additional light source and/or at least one additional photodetector. Each photodetector is separated from a particular light source by a corresponding separation distance. The apparatus further includes a processor configured to collect data based on intensity information collected by the plurality of photodetectors responsive to activation of one or more of the light sources. The data is used to estimate an oxygen saturation level for a layer of placental tissue in a uterus of a mother.

In accordance with at least one embodiment of the first aspect, the apparatus further includes at least one motion sensor configured to collect data indicative of maternal abdominal surface movements including fetal activity or uterine contraction.

In accordance with at least one embodiment of the first aspect, the apparatus further includes a communications interface, and the apparatus is communicatively coupled to an auxiliary device including at least one additional motion sensor.

In accordance with at least one embodiment of the first aspect, data collected from the at least one additional motion sensor is used to filter the data indicative of maternal abdominal surface movements such as fetal activity and uterine contraction collected from the at least one motion sensor of the apparatus.

In accordance with at least one embodiment of the first aspect, the apparatus further includes a temperature sensor configured to collect data related to the body temperature of the mother.

In accordance with at least one embodiment of the first aspect, the processor is further configured to: receive signals from the plurality of photodetectors; and correct the signals using a lookup table that is populated based on a Monte Carlo light transport simulation of a multi-layer tissue sample.

In accordance with at least one embodiment of the first aspect, the processor is further configured to: process the corrected signals in accordance with a machine learning and/or deep learning algorithm.

In accordance with at least one embodiment of the first aspect, the machine learning and/or deep learning algorithm comprises a neural network configured to generate a scalar value or a vector of scalar values indicative of a probability of the mother or a fetus having a potential health condition or conditions.

In accordance with at least one embodiment of the first aspect, the processor is further configured to transmit the collected data to a client device, and the client device is configured to process the data via a machine learning and deep learning algorithm.

In accordance with at least one embodiment of the first aspect, the client device comprises a smart phone or tablet computer wirelessly connected to the apparatus.

In a second aspect of the present disclosure, a system for monitoring a health of a mother or fetus during pregnancy is provided. The system includes: a near-infrared spectroscopy (NIRS) probe comprising a plurality of light sources and a plurality of photodetectors; and a client device communicatively coupled to the NIRS probe. Each photodetector is separated from one of the light sources by a corresponding separation distance. Data collected using the NIRS probe is transmitted to the client device for processing and/or visualization.

In accordance with at least one embodiment of the second aspect, the NIRS probe generates current coupled to a control system enclosed in a housing. The control system includes at least one analog-to-digital converter (ADC) for sampling intensity values of the photodetectors.

In accordance with at least one embodiment of the second aspect, the control system further includes a memory storing a lookup table for correcting the data prior to transmitting the data to the client device.

In accordance with at least one embodiment of the second aspect, the lookup table is populated based on a Monte Carlo light transport simulation of a multi-layer tissue sample.

In accordance with at least one embodiment of the second aspect, the client device is configured to process the corrected data in accordance with a machine learning and/or deep learning algorithm.

In accordance with at least one embodiment of the second aspect, the system further includes a server device, communicatively coupled to the client device via a network, wherein the server device is configured to process the corrected data in accordance with a machine learning and/or deep learning algorithm.

In accordance with at least one embodiment of the second aspect, the machine learning and/or deep learning algorithm is a neural network.

In a third aspect of the present disclosure, a method for monitoring a health of a mother or fetus during pregnancy is provided. The method includes: generating a lookup table based on a Monte Carlo light transport simulation of a multi-layer tissue sample; determining thickness information for one or more layers of tissue using an ultrasound probe; determining intensity information collected using a near-infrared spectroscopy (NIRS) probe including a plurality of light sources and a plurality of photodetectors; and determining an oxygen saturation level of different layers of tissues including at least one of a placenta or one or more other tissues above the placenta based on the thickness information, the intensity information, and the lookup table.

In accordance with at least one embodiment of the second aspect, the method further including analyzing data collected by the NIRS probe using a machine learning and/or deep learning algorithm.

In accordance with at least one embodiment of the second aspect, the machine learning and/or deep learning algorithm processes a set of input data including the oxygen saturation level for the layer of tissue of the placenta. The machine learning and/or deep learning algorithm further processes at least one of the following additional parameters: maternal respiratory functions; maternal cardiac functions and/or blood oxygen saturation level; fetal cardiac functions and/or blood oxygen saturation level; fetal movement; or tissue oxygen saturation level for at least one additional layer of tissue.

### BRIEF DESCRIPTION OF THE DRAWINGS

The systems and methods for monitoring the health of a mother and fetus during pregnancy are described in detail below with reference to the attached figures, wherein:

FIG. 1A is an isometric view of a wearable NIRS device, in accordance with some embodiments of the present disclosure;

FIG. 1B is a top view of the wearable NIRS device of FIG. 1A, in accordance with some embodiments of the present disclosure;

FIG. 1C is a front view of the wearable NIRS device of FIG. 1A, in accordance with some embodiments of the present disclosure;

FIG. 2 is a block diagram of the electrical system included in the NIRS device, in accordance with some embodiments of the present disclosure;

FIG. 3 illustrates a system for monitoring the health of the fetus and mother during pregnancy, in accordance with some embodiments of the present disclosure;

FIG. 4 is a timing diagram of a protocol for collecting data samples using the NIRS device, in accordance with some embodiments of the present disclosure;

FIG. 5A illustrates a process for performing data correction when estimating oxygenation levels in multiple layers of tissue, in accordance with some embodiments of the present disclosure;

FIG. 5B illustrates a process for predicting a health status of a mother or fetus during pregnancy, using a machine learning and/or deep learning model, in accordance with some embodiments of the present disclosure; and

FIG. 6 is a flow chart of a method for monitoring tissue oxygenation levels in the placenta, in accordance with some embodiments of the present disclosure.

### DETAILED DESCRIPTION

A safe, wearable, NIRS device is provided for monitoring the health of a fetus and/or mother during pregnancy. The device includes one or more light sources separated from one or more photodetectors. Optimally, the device is arranged such that there are multiple light source-photodetector pairs with different separation distances. The device is capable of monitoring a variety of parameters including, but not limited to, placental oxygen saturation levels, fetal oxygen saturation levels, fetal heart rate, fetal heart rate variability, maternal oxygen saturation levels (in both blood and tissue (e.g., skin, adipose, etc.), maternal heart rate, maternal heart rate variability, breathing rate, breathing depth, skin temperature, uterine contraction, fetal movement events (monitoring type, duration, and intensity of kicks or other movements), maternal movement activities (monitoring type, duration, and intensity of movements such as steps or acceleration of the core torso), and the like. The combination of a large number of parameters, monitored for longer periods of time compared to conventional techniques using other modalities that are not as mobile, can provide better insight into potential health concerns affecting either the fetus or the mother during the pregnancy.

The device uses NIRS for non-invasive measurements using light in the range of, but not limited to, 650 to 950 nm. Multiple wavelengths of light can be used to measure certain parameters like oxygen saturation level, heart rate, or the like. Use of multiple light sources, at multiple wavelengths, and multiple photodetectors at different separation distances can allow for better insight about oxygen saturation levels of different tissue layers in a multi-layer target (e.g., skin, adipose tissue, uterine wall, placenta, and/or fetus). Compared to other non-invasive modalities, such as functional magnetic resonance imaging (fMRI) and positron emission tomography (PET), NIRS offers unique features including having a higher temporal resolution (on the order of milliseconds) and providing additional spectroscopic information about both oxyhemoglobin and deoxyhemoglobin changes in the patient. The NIRS device is smaller and can tolerate subject motion to a larger extent than fMRI. The smaller nature of the device can also lead to increased adoption by patients, for longer periods of time, due to comfort and ease of use.

The wearable and wireless NIRS device includes a compact control system including a compact control board with electronic components disposed thereon, a flexible substrate with the NIRS probe disposed thereon, and one or more motion sensors (e.g., inertial measurement units, accelerometers, gyroscopes, etc.). The NIRS probe includes one or more light sources and one or more photodetectors. In addition, a frame to hold an ultrasound imaging probe can be embedded in the flexible substrate. In some cases, a healthcare professional may operate an ultrasound probe while the NIRS probe is collecting data for a number of parameters, wherein the ultrasound images can be used to augment the signals collected by the NIRS probe. In some cases, the ultrasound probe is used to measure or estimate a thickness of the various layers of tissue between the NIRS device and the placenta or fetus.

The distance between the light sources and the photodetectors varies, e.g., between 1 mm and 80 mm, which creates both close and far source-detector separation distances that can be used to measure oxygen saturation levels of multiple tissue layers substantially simultaneously. Thus, the NIRS probe is capable of measuring oxygen saturation levels of skin, adipose tissue, or blood of the mother near the surface of the skin, but the NIRS probe can also measure oxygen saturation levels of tissue in the placenta or of the fetus as well. The multiple oxygen saturation level measurements of different tissue layers, when combined with other measurements for other parameters like fetal activity, body temperature, or the like, improves the ability to detect potential health conditions in the mother and/or fetus.

Motion sensors are added to the NIRS probe to collect data related to fetal activity (e.g., movement, kicks, etc.). Additional motion sensors can be communicatively coupled with the NIRS probe to measure movement of the mother unrelated to fetal movement, which can be used to filter the signals from the motion sensors used to detect fetal movement.

The ultrasound probe can also be used to measure a thickness of the tissue layers between the NIRS device and the placenta or fetus including a skin layer, an adipose tissue layer, and a uterine wall layer. Oxygenation calculations based on Monte Carlo light transport simulation of multiple layers are compared to clinical data to correct for errors in the received data due to the variability in characteristics of the tissue layers in different people.

The various parameters collected by the NIRS device can be analyzed with a machine learning and/or deep learning algorithm in order to predict a likelihood of adverse health conditions in the fetus or mother. The overall data acquisition rate of the device can be 10 Hz, 20 Hz, or more, which can capture additional physiological signals such as maternal respiratory and cardiac functions, as well as fetal cardiac functions. The additional signals can provide deeper insight into the health of the mother and/or fetus than is traditionally captured using other modalities.

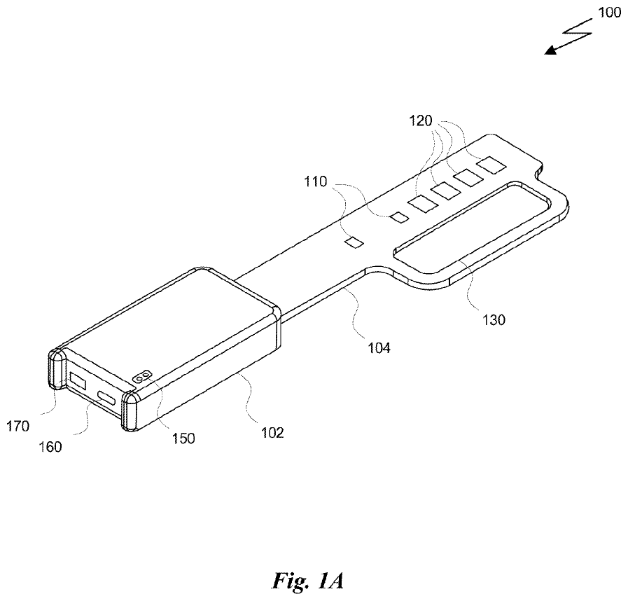

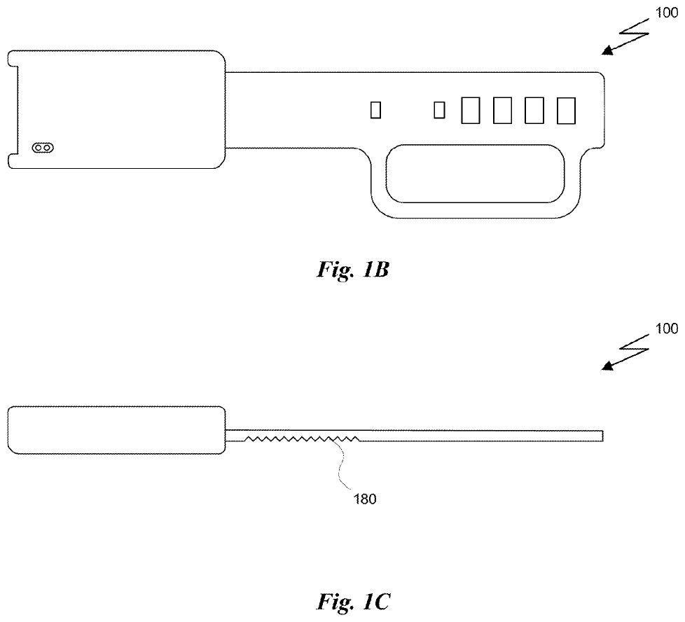

FIG. 1A is an isometric view of a wearable NIRS device 100, in accordance with some embodiments of the present disclosure. The NIRS device 100 includes a housing 102 and a flexible substrate 104 attached to the housing 102. In some embodiments, the housing 102 and flexible substrate 104 are an integrated system manufactured from, e.g., a Rigid-Flex Printed Circuit Board (PCB). In other embodiments, the housing 102 and the flexible substrate are separate components that can be connected or detached via a connector or cable (e.g., an extension cable). When an extension cable is used, the housing 102 may be placed next to the patient, such as on a bedside table, while the NIRS probe embedded in the flexible substrate 104 is worn.

The housing 102 can enclose a number of components including, but not limited to, a control board, a processor, a battery, a memory, a broadband communications chip, a number of sensors, and the like. In an embodiment, the flexible substrate 104 can be a silicone or rubber material that is comfortable when placed against a person's skin. For example, in an embodiment, the flexible substrate 104 can be polydimethylsiloxane (PDMS). The flexible substrate 104 also includes the NIRS probe including one or more light sources 110 and one or more photodetectors 120. There should be at least two different separation distances, so the NIRS device 100 should include, at minimum, a first light source 110, a first photodetector 120, and at least one additional light source 110 and/or at least one additional photodetector 120. In at least some embodiments, the NIRS probe includes two or more light sources 110 and two or more photodetectors 120. The components included in the housing 102 can be programmed to control data acquisition sequences to capture a number of maternal and fetal signals from a plurality of sensors, including the NIRS probe. The measurement results can be uploaded in real time to another host device (e.g., a client device) and/or be transferred to a server device or the cloud for further processing. Post-processing and data visualization may be performed using the host device.

As shown in FIG. 1A, the NIRS probe includes two light sources 110 and four photodetectors 120. Each light source 110 can be a multi-wavelength light source comprising, e.g., a number of light emitting diodes (LEDs) of different wavelength. For example, the light source can include one or more red LEDs at 650 nm wavelength and one or more infrared (IR) LEDs at 850 nm. In an embodiment, each light source 110 may contain a number of LEDs that emit the following wavelengths: 735, 810, and 850 nm. Of course, each light source 110 can include LEDs of other wavelengths as well. Furthermore, the light source 110 can include multiple LEDs of the same wavelength to increase light intensity output at that wavelength. Finally, in some embodiments, the light source 110 can include optical elements such as a light diffusion film, waveguide, lens, or other optical elements to direct the light from the light source towards the target (e.g., the skin).

The NIRS probe also includes one or more photodetectors 120 such as photodiodes, a metal-semiconductor-metal (MSM) photodetectors, or the like. Each photodetector 120 can include a number of sensors (e.g., photodiodes) coupled with different optical filters in order to measure the intensity of light at different wavelengths at a particular location on the NIRS device 100. For example, a particular photodetector 120 can include a first photodiode having an optical filter for a first wavelength located above the first photodiode and a second photodiode having an optical filter for a second wavelength located above the second photodiode. Thus, a single photodetector 120 located at a particular location on the flexible substrate 104 can measure light intensity for two (or more) wavelengths of light from the light source(s) 110. It will be appreciated that a single photodetector 120 can include any number of photodiodes corresponding to one or more wavelengths of light.

Each photodetector 120 is separated from a particular light source 110 by a different separation distance. The different separation distances enable the photodetectors 120 to receive signals from the particular light source 110 reflected from different depths. By using multiple light sources 110 with corresponding multiple photodetectors 120, the NIRS device 100 is capable of receiving a number of signals simultaneously, which can provide insights such as blood oxygenation levels for different tissues of the target located at different depths from the surface of the skin. Thus, signals from different photodetectors 120 can provide oxygen saturation levels of skin, adipose tissue, and placenta at the same time. For example, in an embodiment, the two light sources 110 and four photodetectors 120 are arranged to create six separation distances between 10 and 60 mm. The NIRS probe is also capable of measuring a number of maternal physiological signals based on the signals from the photodetectors 120, including, but not limited to, respiratory functions, cardiac functions, oxygenation levels, or fetal cardiac functions.

The NIRS device 100 can also include a hole 130 for placement of an ultrasound (US) probe. In some embodiments, the hole can include a frame made of plastic and embedded in the flexible substrate 104. The frame can add rigidity to the edge of the hole 130. The location of the hole 130 can be selected to allow for a healthcare professional to capture US images of the placenta or fetus while the NIRS device 100 is being worn by a patient. The US images can also provide information for estimating the thickness of the layers of tissue between the patient's skin and the fetus and/or placenta located directly below the NIRS probe. The estimates may be made with tools for taking measurements using the US probe, such as by selecting two points on a display device used to display the US images with a mouse or other input device. In some embodiments, the US probe may be configured to transmit the measurement information to the NIRS device 100.

The housing 102 can be made of a rigid plastic and encloses various electronic components. The housing 102 can also include various interfaces such as indicator lights 150, a Universal Serial Bus (USB) port 160, and a power button 170 (on/off switch). The indicator lights 150 can provide feedback to a user that the device is on, or provide a charge level of the device. In some embodiments, the housing 102 can also include a display (not shown) such as a liquid crystal display (LCD), LED/OLED display, touchscreen, or the like. The USB port 160 can be used for charging the NIRS device 100 and/or communicating with a host device such as a laptop computer, tablet computer, personal computer, or the like. The power button 170 can be used to turn on the NIRS device 170. It will be appreciated that the NIRS device 100 can include additional interfaces such as additional buttons for initiating or stopping measurements, selecting different operating modes, or the like.

FIG. 1B is a top view of the wearable NIRS device of FIG. 1A, in accordance with some embodiments of the present disclosure. FIG. 1C is a front view of the wearable NIRS device of FIG. 1A, in accordance with some embodiments of the present disclosure. As shown in FIG. 1C, the flexible substrate 104 can include grooves 180 or other features in a portion of the flexible substrate 104 proximate the housing 102 that increase the flexibility of the flexible substrate near the housing 102. This can allow for easier adjustments of the placement of the housing 102 when the NIRS probe is placed on the mothers skin, allowing for more comfort while the device is in use.

In some embodiments, the flexible substrate 104 is long enough to be wrapped around the mother's body and secured to the housing 102, in order to secure the NIRS probe to the mother's skin without requiring any additional means for holding the NIRS probe in place. In other embodiments, the NIRS probe may be held in place against the mother's abdomen by being placed under the mother's clothing or by any other suitable means for holding the NIRS probe against the skin.

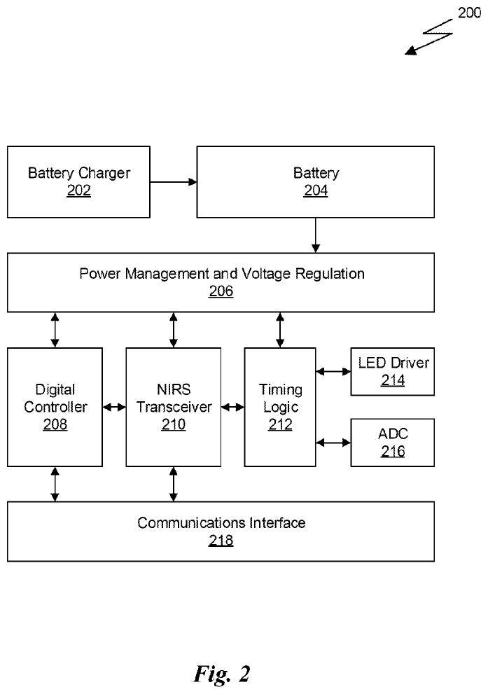

FIG. 2 is a block diagram of the electrical system 200 included in the NIRS device 100, in accordance with some embodiments of the present disclosure. The electrical system 200 can be included in the housing 102 and may be referred to, alternatively, as a control system.

As shown in FIG. 2, the electrical system 200 includes a battery charger 202 connected to a battery 204. The battery charger 202 can be coupled to the power pins of the USB port 160 to provide direct current (DC) power to the battery charger 202 for charging the battery 204. The battery 204 can be a lithium ion battery or any other battery technology capable of storing energy for running the other components of the electrical system 200. Alternatively, a battery may be omitted if the device is plugged in to an external power source, such as through an alternative current (AC) adapter that generates a DC power from a mains electricity of a house or power grid.

The battery 204 can be connected to a power management and voltage regulation 206 chip, sometimes referred to as a power management integrated circuit (PMIC). The battery 204 provides a first voltage to the power management and voltage regulation 206, which regulates one or more voltages to the various components of the electrical system 200. The power management and voltage regulation 206 can enable or disable certain subsystems of the electrical system 200 in order to save energy, as needed, such as operating the NIRS device 100 in various operating modes including a full operation mode, a low power mode, and/or a sleep mode.

The power management and voltage regulation 206 provides supply power to the digital controller 208, the NIRS transceiver 210, the timing logic 212, the LED driver 214, the analog to digital converter (ADC) circuit 216, and/or the communications interface 218. It will be appreciated that some connections between the power management and voltage regulation 206 and the various components or subsystems of the electrical system 200 may not be shown explicitly in FIG. 2.

The digital controller 208 can include, but is not limited to, one or more processors, a digital signal processor (DSP), a microcontroller, a field programmable gate array (FPGA), or the like. The digital controller 208 includes logic to implement the various functions of the NIRS device 100. In some embodiments, the digital controller 208 is an integrated circuit capable of executing instructions embodied in firmware and/or software to implement the functions of the NIRS device 100. The firmware and/or software can include an operating system, drivers, applications, libraries, or the like.

The NIRS transceiver 210 may be connected to the digital controller 208 and includes logic for operating the NIRS probe. For example, each of the light sources 110 can be turned on or off independently. In addition, each of the light sources 110 may be operated at one or more different frequencies and/or intensities. The NIRS transceiver 210 includes logic to configure the parameters of the one or more light sources 210, which are used to generate signals for the timing logic 212. The parameters can include, e.g., driving current, timing signatures, amplification gains, noise reduction filters, and the like. The NIRS transceiver can be configured to upload the captured digital signals (i.e., input data sequences) to a main bus (not explicitly shown) to store the signals in a memory or process the signals via the digital controller 208. The bus may be implemented as a serial parallel interface (SPI) bus or an Inter-Integrated Circuit (I2C) bus, or the like. The NIRS transceiver 210 can be embodied as an application specific integrated circuit (ASIC) and include logic for operating many aspects of collecting the data sequences for a variety of parameters. The NIRS transceiver 210 can operate in conjunction with the digital controller 208.

The timing logic 212 operates in a synchronous time domain as an interface to the LED driver 214, which drives current to the light sources 110. While the digital controller 208 and/or NIRS transceiver 210 may or may not guarantee real-time operation of certain operations scheduled by the instructions or logic (e.g., due to being based on a non-real time operating system like Linux or certain Windows IoT varieties), the timing logic 212 is designed to ensure real-time or on-time operations interfacing with the NIRS probe. For example, a protocol or a timing diagram for sampling a sequence of data using the NIRS probe is illustrated and described in conjunction with FIG. 4, set forth below.

The timing logic 212 can precisely control the gating of current to the one or more light sources 110 by turning on or off the LED driver 214 circuits. The LED driver 214 will generate short pulses of current to each light source sequentially over time with accurate timing control. The LED driver 214 may include separate LED driver circuits for each LED including the one or more light sources 110. Alternatively, each LED driver 214 circuit can be connected to a multiplexor such that LED driver 214 circuit can drive one of a plurality of LEDs sequentially. A protocol for operating the light sources 110 to take measurements is discussed in more detail below with respect to FIG. 4.

Signals from the photodetectors 120 may be connected to the ADC circuit 216, which samples the signal generated by each photodetector 120 sequentially, synchronized with the LED driver 214. Data will be sequentially captured from each photodetector 120 with independent measurement on different light sources 110. The ADC circuit 216 can include a sensing capacitor charged by a current generated by a photodetector 120, an analog-to-digital (ADC) converter configured to convert a voltage of the sensing capacitor into a digital signal, and additional logic (e.g., field effect transistors, resistors, diodes, etc.) used to reset the sensing capacitor and trigger the conversion. The timing logic 212 can control a sampling frequency to measure the intensity of light measured by each of the photodetectors 120. Again, each photodetector 120 can include multiple photodetector sites tuned to one or more frequencies, and each photodetector site can generate a separate and distinct signal connected to the ADC circuit 216. It will be appreciated that the ADC circuit 216 can include multiple instances of separate and distinct ADC circuits 216 such that different photodetector signals can be measured substantially simultaneously. Alternatively, each ADC circuit 216 can be coupled to a multiplexor that is configured to select one of a plurality of signals from the photodetectors 120 to measure sequentially.

The communications interface 218 can be any suitable wired or wireless communications interface, such as, but not limited to, a USB interface, an Ethernet interface, a Bluetooth or other near field communications interface, a Wi-Fi interface, a cellular interface, or the like. The communications interface 218 can enable the NIRS device 100 to communicate with one or more other devices over a communications link. In some embodiments, the communications interface 218 enables communications over a local area network or wide area network such as the Internet.

It will be appreciated that the electrical system 200 can include additional components in lieu of or in addition to the components shown in FIG. 2. Although not shown explicitly, the NIRS device 100 can include one or more memory subsystems connected to the digital controller 208, such as but not limited to a volatile random access memory, a Flash memory, a solid-state drive (SSD), a hard disk drive (HDD), or the like. In addition, the electrical system 200 can be coupled to additional sensors (e.g., motion sensors, temperature sensors, etc.), interface elements (e.g., buttons, switches, etc.), a display device, or the like.

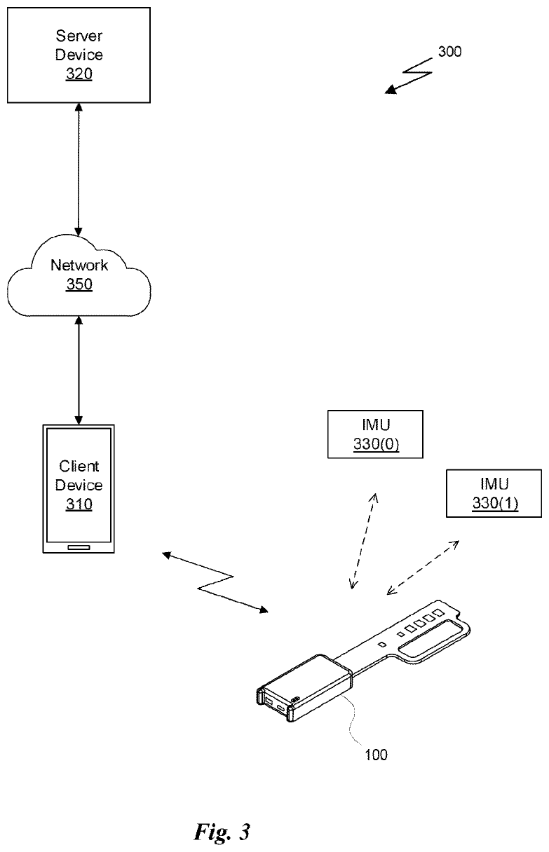

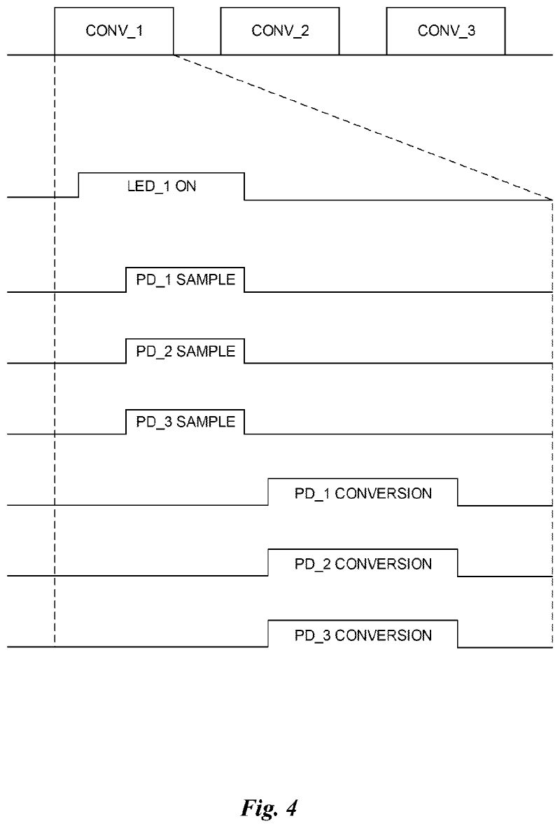

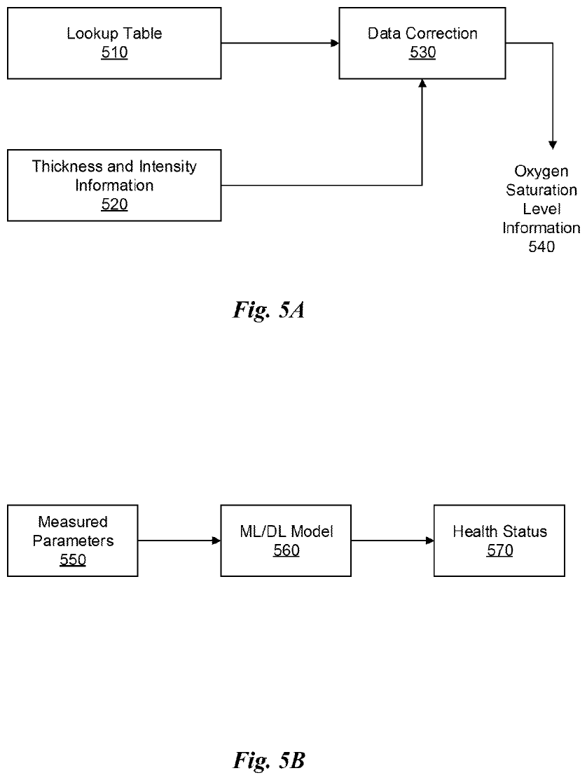

FIG. 3 illustrates a system 300 for monitoring the health of the fetus and mother during pregnancy, in accordance with some embodiments of the present disclosure. The system 300 includes the NIRS device 100, which is communicatively coupled to a client device 310, e.g., via the communications interface 218 of the NIRS device 100. In an exemplary embodiment, the client device 310 can be a smart phone or tablet device configured to communicate with the NIRS device 100 via a Bluetooth wireless communications channel.

The client device 310 can include a client application configured to send control signals to the NIRS device 100 and receive data from the NIRS device 100. The control signals can enable a user of the client device 310 to start or stop measurement collection using the NIRS device 100, change a mode of operation or other parameters of the NIRS device 100, and/or pair the NIRS device 100 with one or more auxiliary devices 330. The client application can also link the data from the NIRS device 100 to a server device 320 via a network 350. The server device 320 can include a server application configured to process the data from the NIRS device 100 to analyze the health of the mother or fetus based on the measured data.

In some embodiments, the NIRS device 100 can be paired with one or more auxiliary devices 330. As shown in FIG. 3, the auxiliary devices 330 can include one or more inertial measurement units (IMU) for measuring motion events unrelated to fetal motion detected by the NIRS device 100 itself. For example, the IMUs 330 can be placed on a limb (e.g., arm, leg, wrist, ankle) to measure motion of the mother that is unrelated to fetal motion. In some cases, the motion data from one or more IMUs 330 can be used to filter motion data collected by an IMU included in the housing 102 of the NIRS device. This filtering can help to isolate motion of the fetus from motion of the mother, thereby providing more accurate information about fetal activity than motion data collected from a single IMU included in the NIRS device 100.

The NIRS device 100 may be paired with a number of auxiliary devices 330 for collecting auxiliary data to increase the capabilities of the NIRS device 100. For example, the NIRS device 100 might be paired with a smart watch capable of collecting ECG/EKG measurements of cardiac activity of the mother, or a blood glucose monitor capable of collecting real-time data related to the mother's blood glucose level. In some embodiments, the data from auxiliary devices is transmitted from the auxiliary device to the NIRS device 100, using the communications interface 218 of the NIRS device 100 and a corresponding communications interface in the auxiliary device 330. The data may then be transferred to the client device 310 from the NIRS device 100. In some embodiments, the NIRS device 100 may process the data from the auxiliary device 330 prior to forwarding the processed data to the NIRS device 100. In yet other embodiments, the auxiliary device 330 may transfer data directly to the client device 310, which then combines the data collected from both the NIRS device 100 and the one or more auxiliary devices 330 for analysis by either the client device 310 and/or the server device 320.

FIG. 4 is a timing diagram of a protocol for collecting data samples using the NIRS device, in accordance with some embodiments of the present disclosure. The principles described below can be adapted for various embodiments where the numbers of light sources 110 and photodetectors 120 vary. Depending on the number of light sources 110 and wavelengths selected, a single sensing period can be divided into a number of different conversion windows. During each conversion window a subset of light sources 110 may be turned on for a first duration of time. The first duration of time should be selected to enable light transmitted through the light source through the tissues of the subject and reflected back to the photodetectors 120. Furthermore, the first duration of time should be selected to allow for enough light to be collected at the photodetector to ensure that the signal to noise ratio (SNR) of the photodetector 120 is acceptable, but not so long as to cause saturation of the photodetector 120. In some embodiments, different photodetectors or photodiodes within each photodetector may have different sensitivities such that if one photodetector or photodiode becomes saturated, a signal from a less sensitive photodetector or photodiode may be used instead. For example, photodetectors 120 with small separation distances can be less sensitive than photodetectors 120 with large separation distances because the intensity of light passing through less tissue is expected to be stronger, given light from the same source at the same intensity. In exemplary embodiments, the first duration of time may be between 50 μs and 75 μs. The first duration of time can be configured by the NIRS transceiver 210 and/or the digital controller 208.

After a delay period, the photodetector signals are sampled. Multiple photodetector signals can be sampled substantially simultaneously during a conversion window. As shown in FIG. 4, three different photodetector signals are sampled. In an embodiment, current from the photodetector 120 is allowed to charge a sensing capacitor during a second duration of time, which may be less than the first duration of time by a delay period. The current signals from the photodetectors 120 are converted to a voltage by the sensing capacitor, and at the end of the first duration of time, the logic turns off the current path to the sensing capacitors such that the voltage of the capacitors is maintained. The light sources 110 can also be turned off once the current path to the sensing capacitors is turned off to save energy and minimize heat transfer from the NIRS probe to the surface of the skin.

Once each of the sensing capacitors has been charged, the voltage of the sensing capacitor can be coupled to an operational amplifier (op amp) to boost the signal to a level that can be accurately measured by the ADC circuit 216 during a third duration of time. The ADC can be supported by a dedicated high speed clock signal provided by the timing circuit 212. The sampled digital signals are transferred to the NIRS transceiver to be stored in a memory and processed by the digital controller 208 or transferred to the client device 310.

In some embodiments, a single conversion window can be used to sample all of the photodetectors 120, as long as the number of hardware resources are sufficient to sample all signals simultaneously. However, in other embodiments, the number of hardware resources available can be less than the desired number of signals to sample. In such cases, multiple conversion windows can be used sequentially to sample signals from different photodetectors or from the same photodetectors but for different wavelengths of light.

In some embodiments, each conversion window is used to measure a single wavelength of light from one or more light sources 110. For example, a first conversion window is used to measure signals from a plurality of photodetectors 120 corresponding to light at 650 nm wavelength. A second conversion window is then used to measure signals from the plurality of photodetectors 120 corresponding to light at 850 nm wavelength.

### Extraction of Maternal and Fetal Physiological Signals

The NIRS device 100 can extract data sequences for a plurality of parameters at data acquisition rates of 10 Hz or higher. NIRS signals can contain, but are not limited to, maternal respiratory functions (e.g., at ~0.3 Hz), maternal cardiac functions (e.g., at ~1 Hz), and fetal cardiac functions (e.g., at ~2.5 Hz). The following describes at least some of the signals captured by the NIRS device for processing and analysis.

Maternal Respiratory Functions—Raw intensity values collected from photodetectors 120 are high-pass filtered at around 0.75 Hz. A peak detection algorithm is applied to find local maximum and local minimum peaks in the filtered signal. Frequency of maximum peaks is corresponding to breathing rate and a difference between maximum and minimum peaks is corresponding to breathing depth. Samples of respiratory parameter signals can be generated continuously or discontinuously at intervals of, e.g., every 30 seconds.

Maternal Cardiac Functions and Blood Oxygen Level—Raw intensity values collected from photodetectors 120 are band-pass filtered from approximately 0.8 Hz to 2 Hz. A peak detection algorithm is applied to find local maximum and local minimum peaks in the filtered signal. Information from the frequency and amplitude of maximum peaks is used to calculate a maternal heart rate, heart rate variability, blood oxygen levels, and other cardiac parameters. In some cases, an electrocardiogram (ECG/EKG) signal of the heartbeat of the mother can be captured and stored.

Fetal Physiological Signals—Fetal heart rate, heart rate variability, and blood oxygenation level can be calculated in a similar way to those maternal signals described above using a different bandwidth of the band-pass and/or high-pass filter to exclude the maternal signals. Based on the data set for extracting fetal physiological signals, the cut-off frequency of the high-pass filter is generally set to 2 Hz, to exclude the maternal physiological signals. Depending on the characteristics of the data, the filter type and cut-off frequency setting may vary.

Fetal Movement Activities-Fetal activity is calculated using 3D information from the one or more motion sensors. Fetal activity is monitored using an IMU in the housing 102 of the NIRS device 100 or embedded within the flexible substrate 104. The signal for fetal activity can be filtered based on motion data from one or more other IMUs detached from the NIRS device 100. Filtering the motion data from the IMU located on the NIRS device 100 based on motion data from an IMU located on an auxiliary device can help to eliminate the effects of maternal movement on the motion data related to fetal activity.

Data from the IMU in the housing 102 can be used to detect at least two types of maternal abdominal surface movements, namely, movements from uterine contraction (e.g., during labor) and movements from fetal movements. During movements due to uterine contraction, movement in the x and y axes may generally decrease while movement in the z axis increases. Furthermore, it has been observed that the magnitude and duration of motion in the z-axis is generally larger during a first contraction than a second or subsequent contraction. Thus, there is potential to measure the strength and length of contractions during labor using the NIRS device 100. During movements due to fetal motion movement in all three axes are generally observed. In general, variation in x, y, and z coordinates of the position of the IMU can be used to monitor fetal movement.

Tissue Oxygenation Levels-Both maternal tissue oxygen saturation levels (e.g., skin, adipose, uterine wall, etc.) and placenta tissue oxygen saturation levels can be measured using the NIRS probe, based on the multiple separation distances of multiple photodetectors with multiple, multi-wavelength light sources. While estimating tissue oxygen saturation level for a single-layer of tissue is performed by some conventional devices, it is more difficult to estimate tissue oxygen saturation level of multiple layers of tissue, where each layer is of unknown and various thickness due to the differences between individuals and at different stages of the pregnancy. The collected intensity information from the photodetectors contains more complex information that must be decoded, as described in the process below.

FIG. 5A illustrates a process for performing data correction when estimating oxygenation levels in multiple layers of tissue, in accordance with some embodiments of the present disclosure. To calculate a single-layer tissue oxygenation level, an intensity of light that has passed through the tissue must be sampled, taking into account the initial intensity of the light source, the measured intensity of the light at the photodetector, and a separation distance between the light source and the photodetector. On the other hand, when light passes through more than one layer of tissue, the light contains complex information about the number of layers of tissue between the light source 110 and the corresponding photodetectors 120. For example, light can be reflected and refracted differently when passing between different boundaries between the layers. Furthermore, each layer can exhibit different characteristics such as transparency, diffusion, scattering, or the like.

In an embodiment, in order to calculate a degree of oxygen saturation level of each layer, a lookup table that considers the thickness of each layer and various optical properties or characteristics of the layers is generated. For pregnant women, in order for light to reach the placenta, the light emitted from a light source 110 must travel through several layers of tissue including skin, adipose tissue, and the uterine wall, each layer having varying thickness on an individual basis as well as for a single individual at different stages of the pregnancy. To address the challenge of accurately estimating tissue oxygen saturation levels in multi-layer tissue, the thickness of all layers can be measured with an ultrasound (US) probe. The measurements from the US probe can then be used as indices into the lookup table for performing data correction calculations.

Taking advantage of the multiple distances between different pairs of light sources 110 and photodetectors 120, the NIRS probe can take measurements corresponding to light traveling through the layers of tissue at different depths. For example, short separation distances between a particular light source 110 and a corresponding photodetector 120 may be used to measure oxygen saturation levels in tissue layers closer to the surface of the skin while longer separation distances between a particular light source 110 and a corresponding photodetector 120 may be used to measure oxygen saturation levels in tissue layers further from the surface of the skin.

It can be difficult to clearly classify each layer of tissue using an ultrasound image. Therefore, in at least one embodiment, the layers of tissue are classified into two groups: a first group representing “epidermis-dermis-adipose” layers and a second group representing “uterus-placenta” layers. A Monte Carlo simulation is used to simulate the complex information contained in the measured signals from the photodetectors 120. The Monte Carlo simulation considers the thickness and optical properties of the two groups of tissue layers classified above, and simulates the expected measured intensities corresponding to different photodetectors for different wavelengths of light by varying the set of parameters associated with the simulation according to a number of distributions.

The Monte Carlo simulation can be performed by taking a number of samples of the set of parameters, simulating the light transport through the tissues based on the samples of the set of parameters, and determining the estimated intensity values collected by each photodetector, given the known timing and intensity of the light sources 110 and separation distances of the photodetectors 120. By taking a number of well-defined single-layer samples and/or multi-layer samples, a distribution of the expected intensity values measured by the NIRS device 100 for different samples of the sets of parameters can be collected and stored in a lookup table. To measure the oxygen level of the mother's placenta using the NIRS device 100, it is necessary to observe the light emitted by the device after the light is scattered and/or reflected by all maternal layers, including the epidermis, dermis, adipose tissue, uterus, and placenta. Each of these layers has different optical properties, and the attenuation degree of the light at the wavelengths of the different light sources (such as 650, 850, and 950 nm) also varies based on these optical properties. Monte Carlo simulation can be used to estimate the intensity of the light measured by photodetectors 120 located at different distances from the NIRS device's 100 light source(s) 110, taking into account the optical properties and thickness of the maternal layers. The lookup table correlates the oxygen levels with the measured thickness of the maternal layer and the measured intensity from the photodetectors 120 at different locations.

In other words, Monte Carlo simulation is used to populate a lookup table based on distributions of a set of parameters that will affect light transport properties in the tissues. Examples of parameters that affect the transport of light include the thickness of each tissue layer, the composition of the tissue layer, the oxygen saturation level of the tissue layer, the wavelength, intensity, and duration of the light source(s), and the separation distance between the light sources and the photodetectors. Some of these parameters are known and fixed (such as the parameters related to the structure of the NIRS device 100 (e.g., separation distance, wavelength, intensity, and duration of light), while other parameters are dependent on the tissue of the subject being measured. However, even the unknown parameters like tissue thickness, tissue composition, and tissue oxygen saturation level can be limited to within a reasonable range and estimated to have a distribution within that range based on experimental measurements of actual tissue. Thus, the light transport simulation may populate the lookup table based on a large number of estimates of the unknown parameters.

As shown in FIG. 5A, a lookup table 510 is populated by performing a Monte Carlo light transport simulation for a multi-layer sample. Data collection 520 is then performed using the NIRS device 100 and an US probe. The data includes measurements for the thickness of the two groups of layers (i.e., thickness information) in the classifications discussed above, using the US probe, and measurements of signal sampled by the photodetectors 120 (i.e., intensity information) in response to light generated by the light sources 110. Data correction 530 is then performed based on the thickness information and the intensity information for each photodetector, given a known separation distance of the photodetector and a corresponding light source of a given wavelength, to generate a corrected oxygenation level for one or more layers of tissue, such as a placenta, skin, or adipose tissue.

FIG. 5B illustrates a process for predicting a health status of a mother or fetus during pregnancy, using a machine learning and/or deep learning model, in accordance with some embodiments of the present disclosure. The process illustrated in FIG. 5A describes one technique for determining an oxygen saturation level in the tissue of a placenta using the NIRS device 100. However, oxygen saturation level of the placenta is merely one parameter for monitoring the health status of the mother or fetus during pregnancy. While this single parameter can be monitored over time, such as by taking a sample measurement every, e.g., N number of seconds either continuously while the NIRS device is in use or periodically every hour, day, or week during pregnancy, additional insight into the health of the mother or fetus can be gained by monitoring multiple parameters over time. For example, oxygen saturation level of the placenta, oxygen saturation level of the skin or adipose tissue of the mother, and oxygen saturation level of the fetus may all be measured using the NIRS device 100 by using signals from different photodetectors having different separation distances. In addition, parameters such as fetal heart rate, fetal heart rate variability, maternal heart rate, maternal heart rate variability, breathing rate, breathing depth, skin temperature, fetal movement events (monitoring type, duration, and intensity of kicks or other movements), maternal movement activities (monitoring type, duration, and intensity of movements such as steps or acceleration of the core torso), and the like can also be collected by the NIRS device 100 and/or auxiliary devices 330 paired with the NIRS device 100. This raw data for a plurality of parameters 550 can then be analyzed by a machine learning (ML) and/or deep learning (DL) model 560 trained to estimate a health status 570 of the mother or fetus.

The ML/DL model 560 can be, e.g., a convolutional neural network (CNN), recurrent neural network (RNN), random forest classifier, ensemble classifier, linear or logistic regression algorithm, support vector machines, or a combination of one or more of the aforementioned algorithms. In an exemplary embodiment, the ML/DL model 560 is a CNN. The CNN includes a number of layers that convert the input data into a scalar value that indicates a health score of the mother or fetus. The health score can be compared against a threshold value. If the health score is greater than a threshold value, then an alert can be set to take remedial action, such as contacting the mother via the client device 310 to encourage the mother to make an appointment with a health care provider, or contacting a server device 320, which sends an alert to a health care provider to contact the mother to schedule an appointment. Other types of remedial action may also be taken by the client device 310 and/or server device 320. Alternatively, an indicator can be displayed on the NIRS device 100, such as by blinking an LED to alert the mother to a potential detected health issue.

In other embodiments, the ML/DL model 560 can be configured as an encoder-decoder framework. The encoder includes a number of layers that convert the input data into a latent space vector or set of vectors. The decoder then converts the latent space representation of the input data into an output. In an embodiment, the output can include a vector of values. Each value in the vector can represent a likelihood or probability that the mother or fetus is experiencing a particular health condition in a plurality of health conditions. As above, each of the values in the vector can be compared against a threshold value to determine whether to set an alert or take other remedial action. The threshold value can be the same for all values in the output or each value in the output can be compared against a different threshold value. The NIRS device 100 combined with an ultrasound probe and Monte Carlo simulation is used to measure/predict the oxygen level considering the thickness of the various layers of maternal tissue (e.g., epidermis, dermis, fat, uterus, and placenta) and the intensity of light sources observed by different location photodetectors. Monte Carlo simulation data is converted into one-dimensional vector and/or array and corrected based on multiple oxygen level results observed by the NIRS device 100 through data matching/correction algorithm of the ML/DL model 560. The ML/DL model 560 models for data matching/correction algorithms adjust the layer structure of the model according to the difference between Monte Carlo simulations and observed results with the NIRS device 100. The result output by the ML/DL model 560 is the oxygen level of each layer of maternal tissue and has the form of a one-dimensional array.

The ML/DL model 560 can be implemented within the NIRS device 100 such as by executing instructions for implementing the ML/DL model 560 using the digital controller 208 (e.g., a processor). Alternatively, the ML/DL model 560 can be implemented on the client device 310 and/or the server device 320, external to the NIRS device 100.

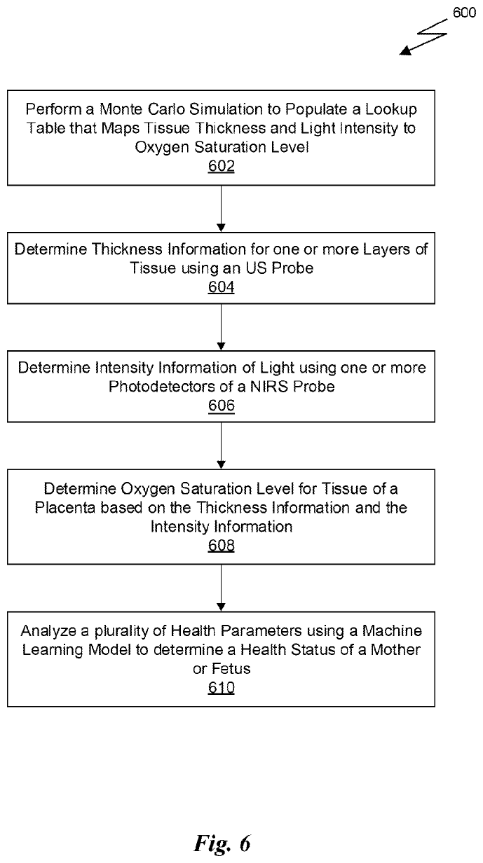

FIG. 6 is a flow chart of a method for monitoring tissue oxygenation levels in the placenta, in accordance with some embodiments of the present disclosure. The method 600 can be performed utilizing the NIRS device 100 of FIGS. 1A-1C.

At 602, a Monte Carlo simulation is performed to populate a lookup table that maps tissue thickness and light intensity to oxygen saturation levels. The lookup table can be used to correct data collected by the NIRS probe using measured thickness information and intensity information.

At 604, thickness information is determined for one or more layers of tissue using an ultrasound probe. In some cases, tissue can be classified into a number of different classification strata, and a thickness of each classification strata is estimated using the US images. 606, intensity information is determined using one or more photodetectors of the NIRS probe. Light intensity measurements corresponding to light from at least one light source, at different wavelengths, can be collected and analyzed to determine the intensity information.

At 608, an oxygen saturation level is determined for tissue of a placenta based on the thickness information and the intensity information. The lookup table can be used to correct the data collected by the NIRS probe, and an oxygen saturation level can be determined based on the corrected data.

At 610, data related to a plurality of health parameters of the mother and/or fetus can be analyzed using a ML/DL model or other traditional computer-implemented algorithms, in conjunction with or in addition to the ML/DL model, to determine or predict a health status of the mother or fetus. The health parameters can include the oxygen saturation level of the placenta as well as additional parameters such as maternal respiratory functions; maternal cardiac functions and/or blood oxygen saturation level; fetal cardiac functions and/or blood oxygen saturation level; fetal movement; or tissue oxygen saturation level for at least one additional layer of tissue. The output of the ML/DL model can be used to detect possible adverse health conditions of the fetus or mother, which can trigger an alert to be issued to the mother or a health care professional.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. Other elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

What is claimed is:

1. An apparatus, comprising:
a light source;
a photodetector;
at least one additional light source and/or at least one additional photodetector; and
a processor configured to collect data based on intensity information collected by the one or more photodetectors responsive to activation of one or more light sources,
wherein each photodetector is separated from a particular light source by a corresponding separation distance; and
wherein the data is used to estimate at least one of an oxygen saturation level for a layer of placental tissue in a mother, or maternal physiological signals including respiratory functions, cardiac functions, oxygenation levels, or fetal cardiac functions.

2. The apparatus according to claim 1, further comprising at least one motion sensor configured to collect data indicative of maternal abdominal surface movements including fetal activity or uterine contraction.

3. The apparatus according to claim 2, further comprising a communications interface, and wherein the apparatus is communicatively coupled to an auxiliary device including at least one additional motion sensor.

4. The apparatus of claim 3, wherein data collected from the at least one additional motion sensor is used to filter the data indicative of fetal activity collected from the at least one motion sensor of the apparatus.

5. The apparatus according to claim 1, further comprising a temperature sensor configured to collect data related to the body temperature of the mother.

6. The apparatus according to claim 1, wherein the processor is configured to:
receive signals from the one or more photodetectors; and
correct the signals using a lookup table that is populated based on a Monte Carlo light transport simulation of a multi-layer tissue sample.

7. The apparatus according to claim 6, wherein the processor is further configured to:
process the corrected signals in accordance with a machine learning and/or deep learning algorithm.

8. The apparatus according to claim 7, wherein the machine learning and/or deep learning algorithm comprises a neural network configured to generate a scalar value or a vector of scalar values indicative of a probability of the mother or a fetus having a potential health condition or conditions.

9. The apparatus according to claim 1, wherein the processor is further configured to transmit the collected data to a client device, and wherein the client device is configured to process the data via a machine learning and deep learning algorithm.

10. The apparatus according to claim 9, wherein the client device comprises a smart phone or tablet computer wirelessly connected to the apparatus.

11. A system for monitoring a health of a mother or fetus during pregnancy, the system comprising:
a near-infrared spectroscopy (NIRS) probe comprising one or more light sources and one or more photodetectors, wherein each photodetector is separated from one of the light sources by a corresponding separation distance; and
a client device communicatively coupled to the NIRS probe,
wherein data collected using the NIRS probe is transmitted to the client device for processing and/or visualization.

12. The system according to claim 11, wherein the NIRS probe generates current coupled to a control system enclosed in a housing, and wherein the control system includes at least one analog-to-digital converter (ADC) for sampling intensity values of the photodetectors.

13. The system according to claim 12, wherein the control system further includes a memory storing a lookup table for correcting the data prior to transmitting the data to the client device.

14. The system according to claim 13, wherein the lookup table is populated based on a Monte Carlo light transport simulation of a multi-layer tissue sample.

15. The system according to claim 14, wherein the client device is configured to process the corrected data in accordance with a machine learning and/or deep learning algorithm.

16. The system of claim 14, the system further comprising a server device, communicatively coupled to the client device via a network, wherein the server device is configured to process the corrected data in accordance with a machine learning and/or deep learning algorithm.

17. The system of claim 16, wherein the machine learning and/or deep learning algorithm is a neural network.

18. A method for monitoring a health of a mother or fetus during pregnancy, the method comprising:
generating a lookup table based on a Monte Carlo light transport simulation of a multi-layer tissue sample;
determining thickness information for one or more layers of tissue using an ultrasound probe;
determining intensity information collected using a near-infrared spectroscopy (NIRS) probe including one or more light sources and one or more photodetectors; and
determining an oxygen saturation level for different layers of tissues such as the placenta and other tissues based on the thickness information, the intensity information, and the lookup table.

19. The method of claim 18, the method further comprising:
analyzing data collected by the NIRS probe using a machine learning and/or deep learning algorithm.

20. The method of claim 19, wherein the machine learning and/or deep learning algorithm processes a set of input data including the oxygen saturation level for the layer of tissue of the placenta, and at least one of the following additional parameters:
maternal respiratory functions;
maternal cardiac functions and/or blood oxygen saturation level;
fetal cardiac functions and/or blood oxygen saturation level;
fetal movement; or
tissue oxygen saturation level for at least one additional layer of tissue.
